(19) United States
(12) Patent Application Publication
(10) Pub. No.: US 2010/0152249 A1
(43) Pub. Date: Jun. 17, 2010

(54) COMPOSITONS AND METHOD FOR TREATMENT OF MOOD AND COGNITIVE IMPAIRMENTS

(75) Inventor: NATALIE L. RASGON, LOS ALTOS, CA (US)

Correspondence Address: Soody Tronson, 2995 Woodside Rd.,, Suite 400, Woodside, CA, 94062, US

(73) Assignee: BOARD OF TRUSTEES OF THE LELAND STANFORD JUNIOR UNIVERSITY, PALO ALTO, CA (US)

(21) Appl. No.: 12/635,103

(22) Filed: Dec. 10, 2009

Related U.S. Application Data
(60) Provisional application No. 61/121,876, filed on Dec. 11, 2008.

(51) Int. Cl. A61K 31/4439; A61K 31/427; A61K 31/421; A61K 31/155; A61P 25/00

(52) U.S. Cl. 514/342; 514/369; 514/374; 514/635

(57) ABSTRACT

Compositions, manufacture, and methods for diagnosis and treatment of subjects exhibiting mood disorders and/or being at risk of cognitive impairment.

### RELATED APPLICATIONS

The present application claims priority to U.S. Provisional Patent Application Ser. No. 61/121,876, entitled Methods and Devices for Use in Diseases of Central Nervous System,” by Rasgon, filed on Dec. 11, 2008.”; the full disclosure of which is incorporated herein by reference.

### FIELD OF THE INVENTION

The present invention is generally related to compounds, compositions, and methods for treatment of mood and or cognitive impairment, and more particularly, in subjects with underlying insulin resistance.

All publications, patents, and published patent applications referred to herein are incorporated herein by reference in their entirety.

### BRIEF DESCRIPTION OF THE DRAWINGS

FIG. 1 is a graphical representation of the HOMA-IR and Cognitive Performance.

FIGS. 2 and 3 are graphical representations of the relationship between BMI and FPI in controls and women subjects with bipolar disorder.

### DESCRIPTION

The present invention is directed to compounds, compositions, and methods for treatment of subjects exhibiting mood disorder; and/or susceptible to or at risk of (e.g., genetic risk for cognitive impairment, as for example by virtue of carrying apolipoprotein ε-4 or having family history of AD) or exhibiting cognitive impairment. In an embodiment the subjects are pre-disposed to (e.g., have surrogate blood markers suggestive of IR) or exhibit insulin resistance (IR). In an embodiment, the subject is exhibiting mood disorder and is at risk of cognitive impairment. The mood disorder may be in remission. In an embodiment, the subjects exhibiting mood disorder and either having or being pre-disposed to IR, are non-diabetic.

Compounds and compositions comprising the same, and within the scope of the present invention, are modulators of insulin resistance, their derivatives and/or salts, and combinations thereof; and are individually or collectively, herein, referred to as insulin sensitizing agents (ISA).

An exemplary method for treating a subject exhibiting mood disorder and/or being at risk of cognitive impairment, comprises identifying a subject exhibiting mood disorder and/or being at risk of cognitive impairment, and administering to the subject a therapeutically effective amount of an insulin sensitizing agent (ISA).

An exemplary method for treating a subject exhibiting mood disorder and/or being at risk of cognitive impairment, comprises identifying a subject exhibiting mood disorder and/or being at risk of cognitive impairment, measuring insulin resistance of the subject, establishing whether the subject's insulin resistance passes a pre-determined threshold insulin resistance level indicative of needing therapy, and administering to the subject a therapeutically effective amount of the insulin sensitizing agent (ISA) if the subject's insulin resistance passes the threshold level.

An exemplary method for diagnosis a subject exhibiting mood disorder and being at risk of cognitive disorder, comprises selecting a subject, measuring the SSPG of the subject, comparing the measured SSPG to a threshold SSPG value indicative of mood disorder and/or cognitive impairment, and determining whether the subject is in need of therapy.

In an exemplary embodiment, the use of an insulin sensitizing agent (ISA) for the manufacture of a medicament for the treatment of a subject exhibiting mood disorder and/or being at risk of cognitive impairment, comprises identifying a subject exhibiting mood disorder and/or being at risk of cognitive impairment, and administering to the subject a therapeutically effective amount of an insulin sensitizing agent (ISA).

In the various embodiments, the subject exhibits mood disorder and is also at risk of cognitive impairment. The mood disorder may be active or in remission. In some embodiments, the subject is predisposed to or exhibits insulin resistance. The ISA may comprise of a PPAR gamma agonist, including thiazolidinedione derivative compounds such as rosiglitazone, pioglitazone, ciglitazone, troglitazone, and englitazone salts or derivatives thereof, or combinations thereof; a O- and N-substituted tyrosine derivative, such as farglitazar, salts or derivates thereof, and combinations thereof. In an embodiment, the ISA may be a biguanide, such as metformin salts or derivates thereof, and combinations thereof.

The compounds of the present invention may also comprise anti-diabetic agents such as insulin, insulin derivatives and mimetics; insulin secretagogues such as the sulfonylureas, e.g., Glipizide, glyburide and Amaryl; insulinotropic sulfonylurea receptor ligands such as meglitinides, e.g., nateglinide and repaglinide.

Mood disorders including major depressive disorder (MDD), bipolar disorder (BD) and diabetes including type II diabetes (DM2), share numerous pathophysiological characteristics that reflect bidirectional links between the central nervous system (CNS) and endocrine homeostasis. Patients with DM2 have a high incidence of depression (Rasgon, N., Jarvik, L., 2004. Insulin resistance, affective disorders, and Alzheimer's disease: review and hypothesis. J Gerontol A Biol Sci Med Sci, 59 (2), 178-183), and reciprocally, patients with depression are at increased risk of developing DM2. Insulin resistance (IR) or “pre-diabetes” is often accompanied by depressive symptomatology and patients with depression have biomarkers suggestive of high IR (Rasgon, N., Jarvik, L., 2004. Insulin resistance, affective disorders, and Alzheimer's disease: review and hypothesis. J Gerontol A Biol Sci Med Sci, 59 (2), 178-183).

Both MDD and DM2 are characterized neurologically by a loss of neuronal integrity in the limbic area (most notably, atrophy of the hippocampus) (den Heijer T, Vermeer S, van Dijk E, Prins N, Koudstaal P, Hofman A et al. Breteler M, Type 2 diabetes and atrophy of medial temporal lobe structures on brain MRI. Diabetologia 2003; 46(12):1604-10). Deficits in memory, attention, and executive function accompany both disorders. Memory and attention appears to improve with glycemic control in patients with DM2.

However, the cognitive deficits observed in patients with MDD remain even after remission of depressive symptoms, possibly due to underlying IR. Further, IR in patients with depression often remains even after remission of depressive symptoms, suggesting a possible bidirectional link between proper insulin function and cognitive abilities. The persistent neuropsychological impairment in patients with remitted or partially-remitted depression may be due in part to underlying untreated impairment in insulin sensitivity, which itself contributes to the onset of depression. Insulin dysfunction in the central nervous system may have detrimental effects on proper serotonin functioning.

As such, it is believed that insulin resistance is a trait marker of depression, rather than a state marker, given that insulin resistance in actively depressed patients persists even after the depressive symptoms have remitted. It is believed by the inventor, without intending any limitations on the scope of the invention, that insulin function is central to proper mood regulation and maintenance of memory and attention.

Alzheimer's disease (AD), a cognitive disorder, is a progressive, debilitating disease and is the most common cause of dementia. Typical symptoms include memory impairment, disordered cognitive function, behavioral changes (including paranoia, delusions, loss of inhibitions) and decline in language function. Pathologically, AD has been traditionally characterized by the presence of two distinct types of brain lesion-neuritic plaques (sometimes referred to as senile plaques) and neurofibrillary tangles.

Mild cognitive impairment (MCI) is a condition in which the subject has a slight impairment in cognitive function that is detectable from their pre-morbid baseline, but which also is not sufficiently severe to fulfill diagnostic criteria for AD. As such, MCI may be considered as a transition state between normal cognitive function in a normal aging subject, and the abnormal cognitive function in dementia. MCI can be subdivided into categories based upon the types of cognitive deficits that are detected. A deficit of memory alone typifies amnestic MCI; whereas other types of MCI involve deficits in multiple cognitive domains including memory, or deficits in a single, non-memory domain. The rate of progression from amnestic MCI to AD has been measured in cohort studies to range from 10-20% per year (Petersen et al. Arch Neurol 2001 58: 1985-1992).

Glucose metabolism is of critical importance in the function of cells within the central nervous system. Decreases in cerebral glucose metabolism that are regionally specific have been demonstrated in patients with AD (Reiman E M et al. New Eng J Med 1996 334: 752-758; Alexander, G E et al. Am J Psychiatry 2002 159:738-745), both in LOAD and in familial AD (Small G W et al., PNAS 2000 97: 6037-6042).

The decrease in cerebral glucose metabolism in patients at risk for AD has been linked to APOE (ApoliPOprotein E) status, because the regionally specific pattern of decreased cerebral glucose metabolism can be detected many years before the predicted age of onset of clinical symptoms, in individuals who carry one or two APOE4 alleles (Reiman E M et al. New Eng J Med 1996 334: 752-758; Rossor M et al., Annals N Y Acad Sci 1996 772:49-56; Small G W et al., PNAS 2000 97: 6037-6042).

Insulin is necessary for glucose utilization in the periphery and for neuronal survival in the CNS. Until more recently, the human brain was considered to be an insulin-insensitive organ. However, clear evidence now shows that insulin has a key role in multiple brain functions, that it is responsible for glucose utilization in the brain, especially in the cortical regions, and that it affects a range of neurotransmitters (i.e. acetylcholine, norepinephrine, and dopamine) that are central in cognitive processes. Insulin influences the release and reuptake of catecholamines and the regulation of regional cerebral blood glow, the autonomic nervous system, and the HPA axis (Davis S, Colburn C Dobbins R, Evidence that the brain of the conscious dog is insulin sensitive. J Clin Invest 1995; 95:593-602). Insulin is necessary for neuronal survival in the CNS. Insulin improves impaired hippocampal long-term potentiation in rodent models of diabetes.

Insulin is also of critical importance in peripheral and central energy metabolism. Secreted by pancreatic β-cells, plasma insulin serves to regulate glucose levels in the blood through periods of feeding and fasting, the rate of glucose uptake in insulin sensitive tissues being controlled by insulin-sensitive glucose transporters. Increases in blood glucose result in the release of insulin, while decreases in blood glucose results in the release of counter-regulatory hormones which increase glucose output by the liver. Type II diabetes results from a reduced ability of insulin to stimulate glucose uptake and to inhibit hepatic glucose output (known as insulin resistance) and an insufficient insulin secretory response to compensate for the insulin resistance.

Insulin is transported across the blood/brain barrier by an insulin receptor-mediated transport process. Peripheral levels of insulin tend to correlate with levels in the central nervous system (CNS), i.e. increased peripheral insulin results in increased CNS insulin. Evidence suggests that insulin has some involvement in normal memory function, and that disorders in peripheral insulin metabolism, such as insulin resistance and hyperinsulinaemia, may have a negative influence on memory. Insulin-promoted increases in glucose utilization may lead to glycolytic production of acetyl-CoA, the key substrate in the synthesis of the neurotransmitter acetylcholine. Reduction in acetylcholine levels is a key feature of AD.

Interestingly, the highest concentrations of neurons that are receptive to insulin are located within the limbic system, especially the hippocampus. These brain regions are particularly important for memory and emotional regulation. In both patients with depression and patients with diabetes, the limbic system brain structures show atrophy and functional activation abnormalities.

It is believed by the inventor, without intending to limit the scope of the invention, that unrecognized, and therefore unresolved IR and subsequent impairment of glucose metabolism in patients with depression may promote neurodegeneration and facilitate the onset of Alzheimer's disease and vascular dementia. Significant evidence exists for the importance of proper, stable glucose metabolism in maintenance of brain function (Convit A, Links between cognitive impairment in insulin resistance: An explanatory model. Neurobiol Aging 2005; 26(Suppl 1):31-5). Insulin resistance has a neurotoxic effect on the hippocampus, potentially mediated through hypercortisolemia, which may be the main mechanism by which changes in endocrine homeostasis affect both mood and cognition.

With IR comes significantly reduced insulin transport into the brain, resulting in CNS glucose deprivation. Since the human brain is almost totally dependent on a continuous supply of glucose, this glucose deprivation likely leads to impairment in brain function. This impairment in brain glucose metabolism can then induce depression and cognition (e.g. memory, attention), which may in turn detrimentally affect behavior that influences quality of glycemic control. Recurrent unavailability of glucose to the brain may have long-term sequelae in the form of recurrent depression or persistent dysthymia (chronic depression), as well as result in cumulative cognitive impairment and increased risk for Alzheimer's disease or vascular dementia. In light of the multiple neuroregulatory functions of insulin in the brain, it may be that CNS insulin deficiency contributes to the evolution and progression of depressive disorders.

Peroxisome Proliferator-Activated Receptor gamma (PPAR-gamma) is an orphan member of the steroid/thyroid/retinoid receptor superfamily of ligand-activated transcription factors. PPAR-gamma is one of a subfamily of closely related PPARs encoded by independent genes (Dreyer C et. Al. Cell 1992 68:879-887; Schmidt A et al. Mol. Endocrinol. 1992 6:1634-1641; Zhu et al. J. Biol. Chem. 1993 268:26817-26820; Kliewer S A et al. Proc. Nat. Acad. Sci. USA 1994 91:7355-7359). Three mammalian PPARs have been isolated and termed PPAR-alpha, PPAR-gamma, and PPAR-delta (also known as NUC-1). These PPARs regulate expression of target genes by binding to DNA sequence elements, termed PPAR response elements (PPRE). To date, PPREs have been identified as the enhancers of a number of genes encoding proteins that regulate lipid metabolism, suggesting that PPARs play a pivotal role in the adipogenic signaling cascade and lipid homeostasis (Keller H et al. Trends Endocrin. Met. 1993 4:291-296).

US Patent Application Publication, 20090192203, the content of which in its entirety is incorporated herein by reference, discloses a class of compounds, Peroxisome Proliferator Activated Receptors (PPARs), which are members of the nuclear hormone receptor super family, which are ligand-activated transcription factors regulating gene expression.

European Patent Patent EP0306228, the content of which in its entirety is incorporated herein by reference, describes a class of PPAR-gamma agonists which are thiazolidinedione derivatives for use as insulin sensitizers in the treatment of Type II diabetes mellitus. These compounds have anti-hyperglycaemic activity. One preferred compound described therein is known by the chemical name 5-[4-[2-(N-methyl-N-(2-pyridyl)amino)ethoxy]benzyl]thiazolidine-2,4-dione and has been given the generic name rosiglitazone. Salts of this compound, including the maleate salt, are described in WO94/05659. European Patent Applications, Publication Numbers 0008203, 0139421, 0032128, 0428312, 0489663, 0155845, 0257781, 0208420, 0177353, 0319189, 0332331, 0332332, 0528734, 0508740; International Patent Applications, Publication Numbers 92/18501, 93/02079, 93/22445 and U.S. Pat. Nos. 5,104,888 and 5,478,852; the contents of all of which in their entirety are incorporated herein by reference; also disclose certain thiazolidinedione PPAR-gamma agonists.

Specific compounds that may be mentioned include 5-[4-[2-(5-ethyl-2-pyridyl)ethoxy]benzyl]thiazolidine-2,4-dione (also known as pioglitazone), 5-[4-[(1-methylcyclohexyl)methoxy]benzyl]thiazolidine-2,4-dione (also known as ciglitazone), 5-[[4-[(3,4-dihydro-6-hydroxy-2,5,7,8-tetramethyl-2H-1-benzopyran-2-yl)me-thoxy]phenyl]methyl]-2,4-thiazolidinedione (also known as troglitazone) and 5-[(2-benzyl-2,3-dihydrobenzopyran)-5-ylmethyl)thiazolidine-2,4-dione (also known as englitazone).

U.S. Pat. No. 6,294,580, the content of which in its entirety is incorporated herein by reference, describes a series of PPAR gamma agonist compounds not of the thiazolidinedione class but which are instead O- and N-substituted derivatives of tyrosine which nevertheless are effective as insulin sensitizers in the treatment of Type II diabetes mellitus. One such compound has chemical name N-(2-benzoylphenyl)-O-[2-(5-methyl-2-phenyl-4-oxazolyl)ethyl]-L-tyrosine (also known as 2(S)-(2-Benzoyl-phenylamino)-3-{4-[2-5-methyl-2-phenyl-oxazol-4-yl)-ethox-y]-phenyl}-propionic acid, or by the generic name farglitazar).

Another class of compounds, biguanides, such as metformin, can also function as oral anti-hyperglycemic drugs used for diabetes mellitus or prediabetes treatment. They are also used as antimalarial drugs. Biguanides do not affect the output of insulin, unlike other hypoglycemic agents such as sulfonylureas and meglitinides. Therefore, not only are they effective in Type 2 diabetics but they can also be effective in Type 1 patients in concert with insulin therapy. Although their mechanism of action may not be fully understood, it is believed that, in hyperinsulinemia, biguanides can lower fasting levels of insulin in plasma. Their therapeutic uses derive from their tendency to reduce gluconeogenesis in the liver, and, as a result, reduce the level of glucose in the blood. Biguanides also tend to make the cells of the body more willing to absorb glucose already present in the blood stream, and there again reducing the level of glucose in the plasma.

The present invention is directed to compounds, compositions, and methods for treatment of subjects exhibiting mood disorder; and/or susceptible to or at risk of (e.g., genetic risk for cognitive impairment, as for example by virtue of carrying apolipoprotein ε-4 or other genes conferring the risk for AD, or having first-degree family member with AD) or exhibiting cognitive impairment. In an embodiment the subjects are pre-disposed to (e.g., have surrogate blood markers suggestive of IR) or exhibit insulin resistance (IR). In an embodiment, the subject is exhibiting mood disorder and is at risk of cognitive impairment. The mood disorder may be in remission. In an embodiment, the subjects exhibiting mood disorder and either having or being pre-disposed to IR, are non-diabetic.

Compounds and compositions comprising the same, and within the scope of the present invention, are modulators of insulin resistance, their derivatives and/or salts, and combinations thereof, individually or collectively, herein, referred to as insulin sensitizing agents (ISA).

The compounds of the present invention may also comprise anti-diabetic agents such as insulin, insulin derivatives and mimetics; insulin secretagogues such as the sulfonylureas, e.g., Glipizide, glyburide and Amaryl; insulinotropic sulfonylurea receptor ligands such as meglitinides, e.g., nateglinide and repaglinide.

Exemplary Studies

In an effort to assess the relationship between distribution of measures of SSPG (Steady-State Plasma Glucose) concentration, which is a state of the art method of measuring insulin resistance, in patients with MDD as compared to healthy age-matched control population, insulin-mediated glucose uptake as assessed by the SSPG concentration in patients with MDD will be measured and compared to values of healthy age-matched control population. It is anticipated that there will be an increase in distribution of measures of SSPG concentration in patients with MDD as compared to healthy age-matched control population.

To assess the association between IR and cognitive performance and clinical course of depression in patients with MDD, the relationship between degree of IR (as evidenced by or the distribution of measures of SSPG concentration) and their neuropsychological performance, may be evaluated.

It is anticipated that there will be statistically significant relationships between degree of IR (as evidenced by or the distribution of measures of SSPG concentration) and neuropsychological performance, i.e., the more insulin resistant an individual (the higher the SSPG concentration), the poorer will be their performance on selected measures of memory and attention. Further, patients with recurrent depression (as indicated by 2 or more major depressive episodes in the lifetime) will have greater IR compared to patients with single episode of MDD.

Preliminary Data

Measurement of Insulin Resistance/Insulin Sensitivity. A variety of measures may be used in studies of IR. The most advanced measurement of IR is via “clamp” procedures that directly measure insulin sensitivity Bloomgarden Z, Measures of insulin sensitivity. Clin Lab Med 2006; 26(3):611-33). Measurements of steady-state plasma glucose (SSPG) using the insulin suppression test has been shown to be highly correlated (r>0.9) with the euglycemic, hyperinsulinemic clamp technique for quantifying insulin-mediated glucose uptake Greenfield M, Doberne L, Kraemer F, Tobey T Reaven G, Assessment of insulin resistance with the insulin suppression test and the euglycemic clamp. Diabetes 1981; 30:387-92).

This procedure is also less burdensome for the patient than the euglycemic, hyperinsulinemic clamp. Indirect, surrogate measures of IR used throughout clinical and epidemiological studies are based on interrelationships between concentrations of insulin and glucose, and in some cases include other parameters obtained either in fasting states or during oral glucose tolerance tests (OGTT) (Radikova Z, Assessment of insulin sensitivity/resistance in epidemiological studies. Endocrine Regulations 2003; 37:189-94).

Compensatory hyperinsulinemia (as demonstrated by elevated insulin levels and normal glucose levels) is observed far earlier than impaired glucose tolerance or overt hyperglycaemia/type II diabetes, thus often going undetected in primary care practice. DM2 can develop when impaired glucose utilization is no longer compensated by increased pancreatic insulin secretion, resulting in overt hyperglycemia (Reaven G, Banting lecture 1988. Role of insulin resistance in human disease. Diabetes 1988; 37(12):1595-1607). Patients with IR are also at greatly increased risk for cardiovascular disease. It is estimated that approximately ⅓ of the healthy, non-obese adult population are insulin resistant, even after controlling for body mass index (BMI). It is also estimated that as many as 50% of individuals with DM2 remain undiagnosed (Mellitus ECotDaCoD, Report of the Expert Committee on the diagnosis and classification of diabetes mellitus. Diabetes Care 2003; 26(suppl 1):S5-S20).

Studies of IR in Patients at Risk of AD.

A study (Rasgon et al, Neurobiology of Aging, 2009) was performed in order to assess the effects of IR on hippocampal volume and cognitive performance in non-diabetic persons at genetic risk for AD (by virtue of carrying apolipoprotein ε-4 or having family history of AD). This point was examined in a cross-sectional fashion using a sample of 50 physically-healthy, cognitively-intact women at genetic risk for AD participating in a larger, longitudinal study evaluating cerebral metabolism, brain structure, and cognitive performance in cognitively-intact, postmenopausal women at risk for AD.

Insulin resistance was characterized by the homeostatic assessment for insulin resistance (HOMA-IR), which is a well-established surrogate biomarker for IR (Hermans, 1999 #1882). HOMA-IR was calculated using the standard formula of HOMA-IR (mM/L×μU/ml)=fasting glucose (mM/L)×fasting insulin (μU/ml)/22.5 (Matthews et al., 1985).

One purpose in identifying modifiable risk factors for AD such as IR, DM2 or cardiovascular disease, was to delay or even prevent neurodegeneration. The results supported this notion by identifying negative effects of IR on hippocampal volumes and neuropsychological performance in cognitively intact middle aged women. This will also be of great use in developing treatments for prospective treatment of depression based on potential changes in IR.

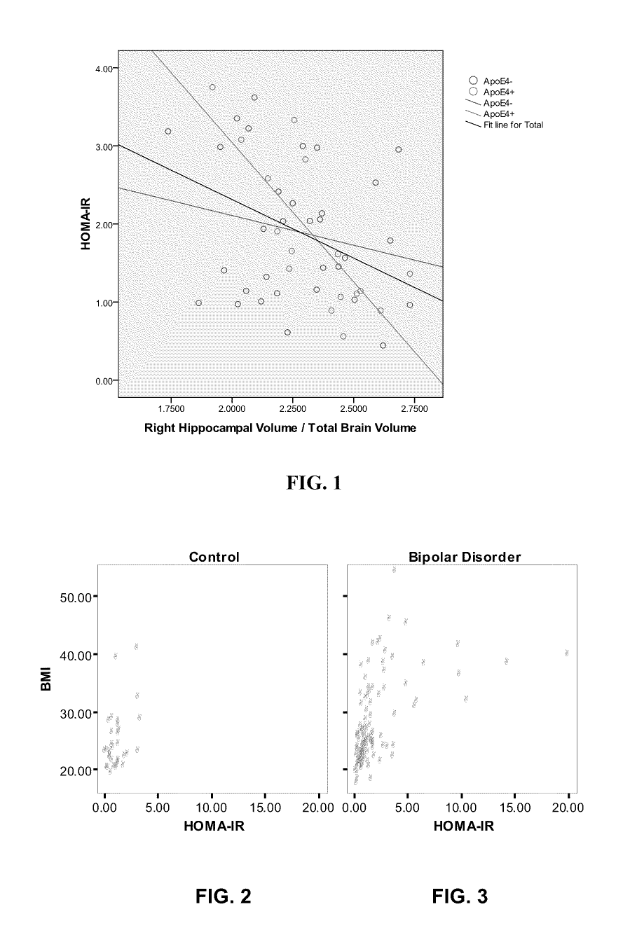

To assess the effect of the improvement of IR with an addition of insulin-sensitizing agent on improving mood in non-diabetic patients with unipolar or bipolar depression who had surrogate blood markers suggestive of IR, the following evaluation was performed. Multiple regression analysis with HOMA-IR and total brain volume as independent variables and left, right, or total hippocampal volume as the dependent variable revealed a significant main effect of HOMA-IR on right hippocampal volume (t=−2.768, p=0.008) and total hippocampal volume (t=−2.367, p=0.022). Given the strong trend correlation between age and right hippocampal volume, age was included with HOMA-IR and total brain volume in a subsequent multiple regression analysis on right hippocampal volume, with results showing a continued significant main effect for HOMA-IR (t=−2.385, p=0.021) but no significant main effect for age (t=−1.423, p=0.162). The data suggested (Table 1, FIG. 1) that IR specifically affects the hippocampus, a structure responsible for memory and other cognitive functions, as there was no association between IR and total brain volume (r=0.137, p=0.342). Results also indicated a significant association between HOMA-IR and cognitive performance on selective tasks, including global cognitive function (MMSE) scores, as well as fluid intelligence (WASI-FSIQ) scores and its subtests.

TABLE 1
Table 1: HOMA-IR and Cognitive Performance

| | Mean (SD) | | Analysis |
|---|---|---|---|
| ACT Total Score | 50.6 | (7.1) | t = −2.202, p = .032 |
| ACT Perseverations | 3.3 | (3.0) | t = 2.829, p = .007 |
| ACT Sequencing Errors | 1.7 | (1.6) | t = 2.801, p = .007 |
| BVRT Total Score | 6.8 | (1.70) | t = −2.494, p = .016 |
| BVRT Errors | 4.9 | (3.0) | t = 2.869, p = .006 |
| BSRT Total Score | 68.3 | (9.9) | t = −1.552, p = .135 |
| BSRT Perseverations | 8.3 | (2.4) | t = −0.349, p = .729 |
| BSRT Deletions | 3.3 | (3.2) | t = 0.082, p = .935 |
| MMSE Score | 29.1 | (0.9) | t = 2.773, p = .008 |
| RCFT Immediate Recall | 21.9 | (5.6) | t = −1.051, p = .298 |
| RCFT Delayed Recall | 21.5 | (5.4) | t = −0.883, p = .382 |
| WASI-FSIQ | 120.5 | (10.3) | t = −3.847, p < .001 |
| WASI Matrix Reasoning | 61.2 | (6.1) | t = −3.953, p < .001 |
| WASI Vocabulary | 61.2 | (7.7) | t = −4.117, p < .001 |

IR in Mood Disorders.

High Rates of IR in Bipolar Depression. A cross-sectional study of metabolic function in women being treated for depression of the bipolar type was conducted. Results showed that 57% of subjects had elevated HOMA-IR values indicative of IR. Further, approximately 31% of women in the sample were overweight (BMI≧25) and 22% were obese (BMI≧30). These findings suggest that IR is common among women being treated for bipolar depression. In a separate small study of women with bipolar depression who were followed longitudinally over a 2-year period, there was a consistent increase in HOMA-IR across all subjects.

Insulin Resistance in Women with Bipolar Disorder. Preliminary analyses from an unrelated prospective controlled study of reproductive and metabolic function in BD suggest that women with BD have significantly higher biomarkers of insulin resistance (IR) and elevated body mass index (BMI) compared to healthy controls. Results are summarized in Table 2.

TABLE 2
Table 2: Women with BD compared to Healthy Control Women

| | BD (n = 103) | Controls (n = 33) | |
|---|---|---|---|
| | mean (SD) | | |
| BMI | 27.7 (7.3) | 24.6 (5.1) | p = .011 |
| Waist circumference | 33.9 (6.6) | 30.8 (4.9) | p = .007 |
| Hip circumference | 41.4 (6.8) | 38.2 (5.0) | p = .015 |
| Waist/hip ratio | 0.82 | 0.81 | p = .406 |
| FPI | 9.6 (9.2) | 6.6 (3.9) | p = .008 |
| FPG | 85.1 (19.0) | 80.2 (13.5) | p = .178 |
| HOMA-IR | 2.21 (2.89) | 1.33 (0.88) | p = .008 |
| TG | 97.8 (74.2) | 76.3 (30.9) | p = .036 |
| HDL | 47.8 (13.1) | 46.0 (14.0) | p = .542 |
| TG/HDL ratio | 2.35 (2.14) | 1.74 (0.81) | p = .034 |

Using a stringent cut-off for HOMA-IR (homeostasis model assessment—insulin resistance) of 3.6, IR was observed in 37.5% of obese BD women, 5.3% of overweight BD women, and 3.9% of normal weight BD women. In contrast, IR was not observed in any of the control subjects. Presence of IR in BD women was statistically significant upon chi-square, at p=0.020.

It is well established in the literature that surrogate indicators of IR, such as HOMA-IR, FPI, TG/HDL ratio, are generally highly correlated with indexes of body weight, such as BMI and waist circumference (WC). Interestingly, in the study's sample of BD women and healthy controls, a greater correlation between these surrogate indicators of IR and BMI/WC among women with BD was observed as compared to controls (see Table 3 and FIGS. 2 and 3 for relationship between BMI and FPI in BD women compared to controls). Further, in a subset (30%) of obese BD women, a disassociation between BMI and HOMA-IR was observed such that some subjects displayed alarmingly high HOMA-IR values with increasing BMI (upper right quadrant of FIG. 1).

TABLE 3
Table 3: Relationship of HOMA-IR
with BMI and WC in BD Women and Controls

| | BMI w/ HOMA-IR | WC w/ HOMA-IR |
|---|---|---|
| BD | r = .499, p < .001 | r = .558, p < .001 |
| Controls | r = .425, p = .015 | r = .358, p = .041 |
| Difference | z = 5.67, p < .001 | z = 4.34, p < .001 |

High Rates of Depression in Women with Polycystic Ovary Syndrome. The frequency of depression among women with Polycystic Ovary Syndrome (PCOS), which is characterized by high rates of IR and obesity, was assessed. In the study, 50% of subjects with PCOS had psychiatric rating scale scores indicative of depression. Those women with depression has significantly higher HOMA-IR values greater IR (p=0.02) and higher BMI (p=0.05) (Table 4). Findings suggest a high frequency of depression among women with PCOS and point to a potential association between depression and biomarkers of IR and metabolic function.

TABLE 4
Table 4: Correlations of Clinical and Biochemical Characteristics
Between Depressed and Non-Depressed Women with PCOS
not being treated with Oral Contraceptives

| | | Non- | | | |
|---|---|---|---|---|---|
| | Depressed | Depressed | | | |
| | women | women | | | |
| Variables | Mean (SD) | Mean (SD) | Statistics | | |
| Log- | 1.71 (0.26) | 1.31 (0.19) | t = −3.10 | df = 13 | p = .01 |
| transformed | | | | | |
| HOMA-IR | | | | | |
| Log- | 3.53 (0.34) | 3.15 (0.29) | t = −2.20 | df = 14 | p = .05 |
| transformed | | | | | |
| BMI | | | | | |

Exemplary Methods for Identification of Subject

In order to assess and directly and qualitatively describe IR and its behavioral and cognitive correlates in MDD patients who are in the remission phase of their depressive impairment, a group of subjects, having the following characteristics will be selected: (a) Men and women ages 30 to 50 years of age; (b) Diagnosis of unipolar, non-psychotic, non-melancholic major depressive disorder (MDD) based on a Structured Clinical Interview for DSM-IV Axis I disorders (SCID) and confirmed by a psychiatrist; (c) Remission as defined by score of <8 on the 21-item Hamilton Rating Scale for Depression, and confirmation of depression remission for at least 3 months by patient's treatment provider; (d) Self-reported subjective cognitive impairment (i.e. memory lapses, sense of reduced cognitive processing speed, etc.); (e) Medically stable (i.e. no uncontrolled or poorly controlled medical illnesses); (f) Adequate visual and auditory acuity to allow neuropsychological testing; (g) Stable regime of antidepressants (with the exception of tricyclic antidepressants) and/or benzodiazepines and over-the-counter medications for at least 6 weeks prior to study procedures, except for medications listed in exclusion criteria. The rationale behind exclusion of tricyclic antidepressants is as follows: these medications are used fairly infrequently and will have less relevance in further studies. In addition, tricyclic antidepressants are implicated in increased IR, which may confound our study results; (g) Cognitively-intact (i.e. no indication of significant deficits upon cognitive testing).

CLINICAL ASSESSMENT. Assessment of medical and psychiatric history, physical and neurological functioning, and hematological, liver, and kidney functioning will be conducted on all subjects at baseline screening.

SCREENING MEDICAL ASSESSMENT. At baseline screening, subjects will undergo a physical and neurological examination and will be asked about personal and family medical history, including any diagnosed medical disorders, both treated and untreated, as well as presence or history of significant central nervous system, respiratory, cardiovascular, urogenital, musculoskeletal, digestive, or endocrine problems. Any significant symptom(s) will be reviewed by the study physician, and if need be, referral for appropriate treatment will be made. Subjects will also be asked about all current prescription and over-the-counter medications, including vitamins and herbal products.

For women, menstrual history (age of menarche, menopausal symptoms and age of onset), pregnancy history, and history of hormonal contraceptives or hormone therapy will be assessed. Lastly, subjects will be asked about family history of dementia, psychiatric disorders, and major metabolic disorders (i.e. diabetes, thyroid disorder). Subjects will also undergo a fasting blood lipid panel (includes total cholesterol, triglycerides, high-density lipoprotein (HDL), low-density lipoprotein (LDL)), a hepatic function panel, and a test of hematocrit level. Electrocardiogram will be conducted as needed to establish patient safety for the SSPG procedure. All results will be reviewed by the study physician, who will then release the results to each participant with instructions to review the results with their primary care physician.

PSYCHIATRIC ASSESSMENT. The psychiatric examination will utilize the Structured Clinical Interview for DSM-IV (SCID; and the 21-item Hamilton Depression Rating Scale (HDRS21). The SCID takes approximately 1-2 hours depending on the pathology of the subject. The SCID had screening and skip-out questions that allow for rapid assessment of Axis I diagnoses. The SCID will be administered in its entirety at the eligibility screening visit.

ANTHROPOMORPHIC ASSESSMENT. Height and weight will be assessed for calculation of body mass index. In addition, waist and hip circumference will be measured. Subjects will complete a detailed diet and exercise questionnaire to assess food intake and energy expenditure, the Pittsburgh Sleep Quality Index (PSQI) to assess sleep quality, and a questionnaire on use of alcohol, cigarettes, nicotine products (i.e. nicotine gums, patches, etc.) to assess intake of alcohol and nicotine. Subjects will complete these questionnaires at the eligibility screening visit.

SALIVARY CORTISOL. Salivary cortisol has been found to be a reliable tool for investigations of hypothalamic-pituitary-adrenal axis activity. Salivary samples will be self-administered in the subjects' own homes, twice a day (waking and 9 pm) on three consecutive days based on established protocol. The average slope between waking and 9 pm will be used as the primary measure of patient cortisol activity. Testing kits will be provided to each participant for the saliva samples. Subjects are given instructions on how and when to collect salivary samples. Samples will be stored at −20° C. until analysis, at which time cortisol will be assayed by enzyme-immunoassay performed by Salimetrics Corp.

ASSESSMENT OF INSULIN SENSITIVITY/STEADY-STATE PLASMA GLUCOSE (SSPG). Insulin-mediated glucose uptake (IMGU) will be quantified by a modification of the insulin suppression test as originally described and validated by Dr. Reaven's research group. After an overnight fast, octreotide will be administered at 0.27 μg/m2/min to suppress endogenous insulin secretion. Simultaneously, insulin and glucose will be infused at 32 mU/m2/min and 267 mg/m2/min, respectively. Blood will be sampled for glucose and insulin levels every half hour until 150 min into the study, and then every ten min until 180 min have elapsed. Insulin concentrations typically plateau by 60 min, whereas glucose concentrations plateau after 120 min. The four values obtained from 150 to 180 min may be averaged and considered to represent the steady-state plasma glucose (SSPG) and insulin (SSPI) concentrations achieved during the infusion. Since SSPI concentrations are comparable in all individuals, both qualitatively and quantitatively, and the glucose infusion rate identical, the magnitude of the resultant SSPG concentration provides an accurate estimate of how effective insulin is in disposal of a glucose load, i.e., the higher the SSPG, the more insulin resistant the person. It should be noted that quantification of insulin action with the IST and the euglycemic, hyperinsulinemic clamp are highly correlated, with a r-value >0.09.

NEUROCOGNITIVE TESTING. Published studies to date suggest that patients with depression demonstrate cognitive deficits most notably in the domains of attention and memory (for review, please see Rogers et al 2004; Marvel & Paradiso, 2004). Therefore, the neuropsychological test battery focuses on these cognitive domains. The following tests will be administered:

Wechsler Abbreviated Scale of Intelligence (WASI; —Selected Subtests). The WASI is a short reliable measure of intelligence in clinical, psychoeducational, and research settings. The vocabulary and matrix reasoning subtests of the WASI allow for measure of general intellectual functioning. The vocabulary subtest is a measure of expressive vocabulary, verbal knowledge, and fund of information. The matrix reasoning subtest is a measure of nonverbal fluid reasoning and general intellectual ability. The data provided from these measures are then used to calculate the Full Scale Intellectual Quotient (FSIQ).

California Verbal Learning Test-2^{nd} Edition (CVLT-II). The CVLT provides a short assessment of the strategies and processes involved in learning and remembering verbal information. It assesses both the amount of verbal material remembered as well as multiple aspects of how verbal learning occurs or fails to occur (both immediate and delayed recall, as well as recognition memory).

Wechsler Memory Scale-III (WMS-III; Selected Subtests). The Logical Memory I and II assesses retention of specific and literal information, as well as more general thematic information, and immediate and delayed recall, and recognition of conceptual material presented in an auditory memory modality (short stories). The Visual Reproduction I and II assess immediate and delayed free recall, as well as recognition memory of two-dimensional visual material.

Delis-Kaplan Executive Functioning System (D-KEFS; selected subtests). The Trail Making Test is a visual motor task comprised of five conditions requiring the examinee to either cross out stimuli or draw trails. It separately assesses visual scanning, number sequencing, letter sequencing, and motor speed that can be utilized to parcel out deficits in these areas from the primary executive function test of cognitive flexibility. The Verbal Fluency Test includes three conditions requiring the examinee to produce as many words possible in a one-minute time period. Tasks assess phonemic (letter) fluency, semantic (category) fluency, and a verbal set-switching (category-switching).

The Color-Word Interference Test consists of four conditions that assess naming and reading speed, verbal inhibition, and cognitive flexibility. Performances on naming and reading tasks are used to parcel out the higher level executive functions of inhibition and cognitive flexibility. The Mesulam Cancellation Task consists of a paper-and-pencil task using verbal and non-verbal stimuli presented both in structured and unstructured arrays requiring the examinee to cancel all of the targets on the page. It is thought to assess a variety of constructs including efficacy and speed of visual scanning, sustained and selective visual attention, concentration, response activation and inhibition.

Wechsler Adult Intelligence Scale-3rd Edition (WAIS-III; selected subtests). The Digit Span Subtest assesses working memory and attention. Number series are presented orally that the examinee is required to repeat verbatim for Digits Forward and in reverse order for Digits Backwards. The Letter-Number Sequencing assesses working memory and attention. Sequences of numbers and letters are presented orally and the examinee is required to track then repeat the numbers in ascending order followed by the letters in alphabetical order.

Statistical Analysis. To assess the following, (1) the relationship between distribution of measures of SSPG concentration in patients with MDD as compared to healthy age-matched control population; and (2) association between IR and cognitive performance and clinical course of depression in patients with MDD; the descriptive statistics and plots of the SSPG data for skewness and outliers will be studied. If these variables do not meet parametric assumptions, the values may be transformed or non-parametric methods used. When appropriate, Bonferroni-type adjustments for multiple tests within domains will be applied in order to limit the Type 1 error risk. As Type II error risk is also of concern, analyses where the critical effect size is unknown have been explicitly designated as exploratory.

To test the first anticipated result that there will be a shift in the distribution of measures of SSPG concentration, with significantly higher values at any degree of adiposity in patients with MDD as compared to the age-matched healthy controls, first t-tests will be applied to test difference in SSPG mean between depressed patients and controls as a whole. Next, multiple regression will be utilized to control for the potential effects of BMI, age, and gender with respect to differences between depressed patients and controls. Lastly, depressed patients according to SSPG tertile cut-offs, will be categorized (McLaughlin T, Allison G, Abbasi F, Lamendola C Reaven G, Prevalence of insulin resistance and associated cardiovascular disease risk factors among normal weight, overweight, and obese individuals. Metabolism 2004; 53(4):495-99) in order to examine the distribution of SSPG relative to BMI.

To test the second anticipated result that there will be statistically significant relationships between degree of IR (as evidenced by SSPG concentration) and neuropsychological performance, Pearson correlations between SSPG concentration and performance on each measure of memory and attention will be examined. Secondly, multiple regression analyses will be utilized to control for the potential effects of age and gender. To examine the potential difference in IR between patients with recurrent depression (as indicated by 2 or more major depressive episodes in the lifetime) compared to patients with single episode of MDD, firstly t-tests, and secondly multiple regression will be applied to control for the potential effects of age and gender.

Based on the above analyses of the study's primary anticipated result, it is determined that cohort of 90 depressed patients and 250 age-matched healthy controls will yield at least 80% power to detect moderate to large effects using a 5% two-tailed test. Power calculations are based on an estimated difference of 40 mg/dL (s.e.=17.0) between depressed patients and healthy controls, obtained by calculating the differences in indirect measures of IR (e.g. HOMA-IR) between patients with depression and healthy controls in previous studies, and the correlation between HOMA-IR and SSPG. Overall, a greater than 80% power is anticipated to detect an effect of depression diagnosis on the independent variable whenever depression diagnosis accounts for 6% or more of the total variance in our sample (i.e. R²=0.06). Given that HOMA-IR has more noise than SSPG, it is expected that the use of SSPG in the present study will increase the signal to noise ratio and further increase power. Confidence intervals and effect sizes will be calculated for all analyses.

The relationship between directly measured IR and cortisol, the latter of which has long been implicated in the pathophysiology of depression may be examined. Interactions between these variables and neuropsychological performance on select measures of memory and attention may also be explored using multiple regression analyses and structural equation modeling.

Exemplary Methods of Treatment

“Treat”, “treating,” and “treatment” refer to a method of preventing, alleviating, abating, ameliorating, a disease and/or its attendant symptoms.

Use of ISA in Treatment of Depressive Disorder.

Open-label PPAR agonist, rosiglitazone, titrated to a dose of 8 mg/day, was administered for 12 weeks to patients with depressive disorder receiving treatment as usual (TAU). Subjects were evaluated and surrogate IR blood markers were chosen based on fasting plasma glucose >100 mg/dL or triglyceride (TG) to high density lipoprotein (HDL) ratio of about 3.0 or greater.

Eight patients who completed the 12 week study exhibited significant declines in both depression severity by Hamilton Depression Rating Scale and clinical assessment (CGI) scores with moderate effect sizes.

Psychiatric Response to Treatment. Repeated measures ANOVA on HDRS-21 data showed a significant decline from a baseline mean of 19.9 to a post-treatment mean of 12.1 (F[2,14]9.103, p=0.019). CGI-S also declined significantly from a baseline mean of 4.0 to a post-treatment mean of 2.9 (F2,14]=7.273, p=0.043). The trajectory of HDSR-21 and CGI score changes are displayed in FIGS. 1 and 2. Cohen's d for pre- to post-treatment HDRS-21 was 1.17, with an effect size of r=0.504. For CGI-S scores, Cohen's d and effect size r for CGI-S were 1.27 and 0.54, respectively. Patients showed a mean 39.3% reduction in HDRS-21 scores, with 4 of the 8 patients showing ≧50% score reduction. Even considering the very small sample size, the trajectory of decline in HDRS and CGI was linear.

These results suggest the efficacy for use of an insulin sensitizing agent in the treatment of depressive disorders.

### CONCLUSION

While this application describes certain exemplary embodiments of compositions, manufacture of the same, and methods for diagnosis and treatment of subjects exhibiting mood disorders and/or being at risk of cognitive impairment, only the attached claims define the scope of the invention.

That which is claimed:

1. A method for treating a subject exhibiting mood disorder and/or being at risk of cognitive impairment, comprising:
Identifying a subject exhibiting mood disorder and/or being at risk of cognitive impairment; and
Administering to the subject a therapeutically effective amount of an insulin sensitizing agent (ISA).

2. The method of claim 1, wherein the subject exhibits mood disorder and is at risk of cognitive impairment.

3. The method of claim 1, wherein the subject exhibits both mood disorder and cognitive impairment.

4. The method of claim 1, wherein the mood disorder is in remission.

5. The method of claim 2, wherein the mood disorder is in remission.

6. The method of claim 1, wherein the subject is predisposed to insulin resistance.

7. The method of claim 2, wherein the subject is predisposed to insulin resistance.

8. The method of claim 2, wherein the subject exhibits insulin resistance.

9. The method of claim 1, wherein the ISA comprises a PPAR gamma agonist.

10. The method of claim 9, wherein the PPAR gamma agonist comprises a thiazolidinedione derivative compound.

11. The method of claim 10, wherein the thiazolidinedione derivative compound is selected from the group consisting of one or more of rosiglitazone, pioglitazone, ciglitazone, troglitazone, and englitazone.

12. The method of claim 9, wherein the PPAR gamma agonist comprises a O- and N-substituted tyrosine derivative.

13. The method of claim 12, wherein the O- and N-substituted tyrosine derivative is selected from the group consisting of farglitazar.

14. The method of claim 1, wherein the ISA is a biguanide.

15. The method of claim 14, wherein the biguanide is selected from the group consisting of metformin.

16. A method for the treatment of a subject exhibiting mood disorder and/or being at risk of cognitive impairment, comprising:
Identifying a subject exhibiting mood disorder and/or being at risk of cognitive impairment;
Measuring insulin resistance of the subject;
Establishing whether the subject's insulin resistance passes a pre-determined threshold insulin resistance level indicative of needing therapy; and
Administering to the subject a therapeutically effective amount of an insulin sensitizing agent (ISA) if the subject's insulin resistance passes the threshold level.

17. The method of claim 16, wherein the subject exhibits mood disorder and is at risk of cognitive impairment.

18. The method of claim 17, wherein the mood disorder is in remission.

19. The method of claim 17, wherein the subject exhibits cognitive impairment.

20. A method for diagnosing a subject exhibiting mood disorder and being at risk of cognitive disorder, comprising:
Selecting a subject;
Measuring the SSPG of the subject;
Comparing the measured SSPG to a threshold SSPG value indicative of mood disorder and/or cognitive impairment; and
Determining whether the subject is in need of therapy.
